(19) United States
(12) Patent Application Publication
(10) Pub. No.: US 2006/0222673 A1
(43) Pub. Date: Oct. 5, 2006

(54) INJECTABLE PHOSPHATIDYLCHOLINE PREPARATIONS

(75) Inventors: Wendy Chern, Harleysville, PA (US); Vijendra Nalamothu, Lansdale, PA (US)

Correspondence Address: ROSS J. OEHLER;SANOFI-AVENTSI U.S. LLC, 1041 ROUTE 202-206, MAIL CODE: D303A, BRIDGEWATER, NJ, 08807, US

(73) Assignee: Aventis Pharmaceuticals Inc., BRIDGEWATER, NJ (US)

(21) Appl. No.: 11/424,952

(22) Filed: Jun. 19, 2006

Related U.S. Application Data
(63) Continuation of application No. PCT/US04/43484, filed on Dec. 21, 2004.
(60) Provisional application No. 60/531,646, filed on Dec. 22, 2003.

(51) Int. Cl. A61K 31/685; A61K 31/56

(52) U.S. Cl. 424/401; 514/171; 514/78

(57) ABSTRACT

The present invention relates to viscous injectable phosphatidylcholine preparations and their use for the reduction or removal of localized adipose tissue (fat) deposits, and to intra-fat pad injection and implant methods of administering such preparations for the non-surgical removal or reduction of localized fatty deposits.

### CROSS REFERENCE TO RELATED APPLICATIONS

This application is a continuation of PCT/US2004/04384, filed Dec. 21, 2004, which claims priority U.S. Provisional Application No. 60/531,646, filed Dec. 22, 2003.

### BACKGROUND OF THE INVENTION

This invention relates to viscous injectable phosphatidylcholine preparations and their use for the reduction or removal of localized adipose tissue (fat) deposits, and to intra-fat pad injection and implant methods for administering such preparations for the non-surgical removal or reduction of localized fatty deposits.

Lipostabil® intravenous solution (containing 93% 3-sn-phosphatidylcholine) is an injectable composition which was developed for the treatment of lipid atheromas, hypercholesterolemas, fat embolisms, and plaque adhering to arterial walls. It is marketed as Lipostabil® N i.V. (Rote Liste, March 2003), and is identified as containing phospholipids, bile acid, DL-alpha-tocopherol, ethanol and water and is approved for the prophylaxis and treatment of fat embolisms. An article in Harper's Bazaar, October 2001, page 137, reported that Lipostabil® solution could be used to reduce subcutaneous fat deposits. However, the Lipostabil® solution used in these reported uses has been the solution that was developed for use as an intravenous injection only, and not for reduction of subcutaneous fat deposits.

It is further reported that fat pads like those occurring in overweight people underneath the eyes, on the abdomen or on the hips shrink, and esthetic improvements in the appearance of the treated people are said to occur when these people received subcutaneous injection of Lipostabil® N i.V. (Patricia Guedes Rittes, The Use of Phosphatidylcholine for Correction of Lower Lid Bulging Due to Prominent Fat Pads, Dermatol. Surg. 2001; 27:391-392).

Aqueous phospholipid liposomal systems are also known for various applications. These systems are employed, for example, in the cosmetic sector or for producing pharmaceutical products. These systems are distinguished by having spherical vesicles which are also referred to as liposomes. The boundary of said liposomes to the outside is formed by a lipid bilayer membrane, and they contain an aqueous phase inside. Aqueous phospholipid systems comprising at least one phospholipid, at least one bile aced and water are described for example in U.S. Pat. No. 6,663,885.

### SUMMARY OF THE INVENTION

The present invention provides a viscous formulation which may be used for intra-fat pad application to reduce or remove localized fat without adverse effects, or at least a reduced level of effects as compared to such use of Lipostabil® solution. Compositions of phosphatidylcholine and functional excipients have been formulated to deliver and maintain a desired concentration of phosphatidylcholine at the targeted sites of the treatment.

The compositions of the present invention comprise about 0.1% to about 25% by weight (w/w) phosphatidylcholine, preferably in an aqueous solution. Lipostabil® solution comprises about 6% w/w phosphatidylcholine in an aqueous solution. In accordance with one embodiment of the invention, a phosphatidylcholine composition is provided which has a viscosity greater than that of Lipostabil® solution.

In accordance with another embodiment of the invention, a phosphatidylcholine composition is provided which has a viscosity greater than that of Lipostabil® solution by incorporating a water soluble thickener or viscous solvent into the composition.

In accordance with a further embodiment of the invention, there is provided a method for reducing subcutaneous fat deposits in a patient comprising the intra-fat pad administration of a composition of the present invention.

### DETAILED DESCRIPTION

The compositions of the present comprise phosphatidylcholine in a formulation suitable for intra-fat pad administration. That is, the composition may be injected through the skin directly into targeted fat pads. Suitable compositions include aqueous solutions, but non-aqueous compositions may also be used. Although the phosphatidylcholine included in the Lipostabil® product is derived from soya beans, there are other sources of phosphatidylcholine available. These include egg-yolk derived phosphatidylcholine and synthetic phosphatidylcholines, such as 1-palmitoyl-2-oleoyl-sn-glycero-3-phosphocholine. The activity of the phosphatidylcholine is not expected to vary significantly whether it is synthetic or derived from soya beans or egg yolk. The compositions of the present invention may include any form of natural or artificial phosphatidylcholines.

Lipostabil® product also comprises deoxycholic acid, which is a well-known bile acid. The compositions of the present invention contain a bile acid, such as deoxycholic acid, in combination with the phosphatidylcholine. Other bile acids include cholic acid and lithocholic acid. The bile acid may also be provide as a salt. Well-known bile acid salts include Na taurocholate, Na taurochenodeoxycholate, Na taurodeoxycholate, and glycodeoxycholate. In one embodiment, the ratio of phosphatidylcholine to bile acid is in the range of about 1 to about 4 w/w. In a particular embodiment, the bile acid used is deoxycholic acid. A particular embodiment of the present invention contains a ratio of phosphatidylcholine to deoxycholic acid of about 2.2 to 1 w/w, which is about the same ratio of these components in Lipostabil® solution.

The composition may also contain pharmaceutical excipients as necessary. In the Examples presented below, the compositions contain benzyl alcohol as a preservative. They also contain sodium chloride as an osmosity adjusting agent. Sodium hydroxide is added to adjust the pH to a desired level for use in a patient.

A solution of Evans blue dye (1% solution in sterile PBS) was injected into abdominal fat pads of B6.V-Lep^{ob} mice to observe its distribution after injection. Immediately after injection, the blue color solution spread the entire abdominal area. To increase the contact time of phosphatidylcholine at the treatment (injection) sites, it is desirable to formulate more viscous solutions. This can be accomplished by increasing the concentration phosphatidylcholine in the solution or by formulating with thickening excipients or using a viscous solvent. Such thickening excipients include, but not limited to, hypromellose (another name for hydroxypropyl methyl cellulose or HPMC), methylcellulose, polyvinyl alcohol or polyvinylpyrrolidone, and viscous solvents include, but not limited to, mineral oil or polyethylene glycols.

As an alternative to direct injections into fat pads, the composition of the present invention can be administered by targeted delivery using in situ gel implantation.

Another embodiment is to administer phosphatidylcholine entrapped in a polymer carrier such as, but not limited to, poly (DL-lactide-co-glycolide); poly(lactide-co-glycolide); poly(DL-lactide); poly(L-lactide); poly(glycolide); poly(ε-caprolactone); poly(DL-lactide-co-caprolactone).

The concentration on in the commercial Lipostabil® product was designed for intravenous application to solubilize fatty matters that are loosely adhered to the vasculature, or dispersed the blood stream. A dose of this product comprises 250 mg phosphatidylcholine in 5 mL. PPC-P is a (3-sn phosphatidyl) choline from soya beans. A higher solubilizing strength is believed to be required for better efficacy against localized fat deposits. Injectable formulations with higher concentrations of phosphatidylcholine (up to 25.0% w/w) can be accomplished, and examples of a few prototypes are given in section 4.

The composition of the present invention can be used to treat a number of disorders including, but not limited to, lipoma, eye bulging, xanthelamas, buffalo neck (also known as buffalo-hump, a condition which occurs in patients due to a redistribution of neck fat) and other rarer diseases of fatty tissue such as fat deposits resulting from Dercum's disease and Launois-Cléret syndrome.

Localized fat deposits, especially related to diseases such as Dercum's and buffalo-hump, are painful and cause discomfort. Such conditions may lead a patient undergo excision procedures such as a liposuction or dermolipectomy. The composition of the present invention is a formulation designed for use in a method of treatment which addresses such subcutaneous fat in a safe and efficacious method. The dose-optimized formulation of the present invention has an appropriate viscosity to deliver and confine the fat-solubilizing composition in the targeted site of treatment. This provides a predictable, consistent and controlled treatment method. In addition, an implantable formulation, releasing the composition over a period of time, such as several weeks, can reduce the number of patient visits and/or number of administrations.

### EXAMPLES

The following examples represent formulation suitable for use as injectable or implantable preparations of phosphatidylcholine. Soya phosphatidylcholine is used in these examples, but phosphatidylcholine of other biological or synthetic sources could be used. The solution viscosity and concentrations of phosphatidylcholine were altered to achieve an improvement in the localized and sustained fate reducing or removing effects at the intended treatment sites.

TABLE 1

| | Example #1 | Example #2 | Example #3 |
|---|---|---|---|
| Ingredient | (% w/w) | (% w/w) | (% w/w) |
| PPC-P | 6.578 | 10.764 | 14.950 |
| Benzyl Alcohol | 0.900 | 0.900 | 0.900 |
| Deoxycholic Acid | 2.939 | 4.810 | 6.680 |
| Sodium Chloride | 0.350 | 0.350 | 0.350 |
| Sodium Hydroxide | 0.268 | 0.268 | 0.268 |
| Sterile Water for | Q.S. 100.00 | Q.S. 100.00 | Q.S. 100.00 |
| Injection | | | |

TABLE 2

| | Example #4 | Example #5 | Example #6 | Example #7 |
|---|---|---|---|---|
| Ingredient | (% w/w) | (% w/w) | (% w/w) | (% w/w) |
| PPC-P | 5.980 | 5.980 | 5.980 | 5.980 |
| Benzyl | 0.900 | 0.900 | 0.900 | 0.900 |
| Alcohol | | | | |
| Deoxycholic | 2.672 | 2.672 | 2.672 | 2.672 |
| Acid | | | | |
| Sodium | — | — | — | — |
| Chloride | | | | |
| Sodium | 0.268 | 0.268 | 0.268 | 0.268 |
| Hydroxide | | | | |
| Hypromellose | 0.500 | 0.750 | 0.500 | 0.100 |
| Sterile | Q.S. 100.00 | Q.S. 100.00 | Q.S. 100.00 | Q.S 100.00 |
| Water for | | | | |
| Injection | | | | |
| Viscosity* | 57.6-58.5 | 157-158 | 46.6-48.1 | 5.01-7.01 |
| (cps) | | | | |

*Viscosity of Lipostabil ® solution is 2.00 to 5.01 cps

Viscosity measurements were taken by Brookfield viscometer (Digital viscometer model # DV-II) using spindle#2 at 60 RPM (rotational speed) for 2 minutes.

What is claimed is:

1. An aqueous composition suitable for reducing subcutaneous fat deposits comprising, by weight, about 0.1% to about 25% phosphatidylcholine, a bile acid or bile acid salt wherein the ratio of phosphatidylcholine to bile acid or bile acid salt is between about 1 w/w and 4 w/w, and a thickening agent.

2. The composition of claim 1 wherein the thickening agent is one or more of hypromellose, methylcellulose, polyvinyl alcohol or polyvinylpyrrolidone.

3. The composition of claim 1 wherein the thickening agent is a viscous solvent.

4. The composition of claim 1 wherein the bile acid or bile acid salt is selected from deoxycholic acid, cholic acid, lithocholic acid, Na taurocholate, Na taurochenodeoxycholate, Na taurodeoxycholate or glycodeoxycholate.

5. The composition of claim 1 wherein the phosphatidylcholine is synthetic or derived from natural sources.

6. The composition of claim 5 wherein the phosphatidylcholine is derived from soya beans or egg yolk.

7. The composition of claim 5 wherein the phosphatidylcholine is 1-palmitoyl-2-oleoyl-sn-glycero-3-phosphocholine.

8. The composition of claim 1 wherein the composition contains at least about 6% w/w phosphatidylcholine.

9. A method of reducing subcutaneous fat deposits comprising the administration of the composition of claim 1.

10. The method of claim 9 wherein the administration is by intra-fat pad injection.

11. The method of claim 9 wherein the administration is by in situ gel implantation.
